(19) United States
(12) Patent Application Publication
(10) Pub. No.: US 2025/0127805 A1
(43) Pub. Date: Apr. 24, 2025

(54) ALCOHOLIC FORMULATION WITH A MIXTURE OF ALKALOIDS, PRENOL LIPIDS AND FLAVONOIDS

(71) Applicant: PONTIFICIA UNIVERSIDAD JAVERIANA, Bogota (CO)

(72) Inventors: Susana Fiorentino Gomez, Bogota (CO); Paola Veronica Lasso Apraez, Bogota (CO); Geison Modesti Costa, Bogota (CO); Luis Carlos Chitiva Chitiva, Bogota (CO); Laura Yinneth Rojas, Bogota (CO); Claudia Uruena, Bogota (CO); Natalia Murillo, Bogota (CO)

(21) Appl. No.: 18/918,431

(22) Filed: Oct. 17, 2024

Related U.S. Application Data
(60) Provisional application No. 63/544,727, filed on Oct. 18, 2023.

(51) Int. Cl. A61K 31/7048; A61K 31/015; A61K 31/165; A61K 31/4525; A61K 47/10

(52) U.S. Cl. CPC A61K 31/7048; A61K 31/015; A61K 31/165; A61K 31/4525; A61K 47/10

(57) ABSTRACT

The present invention is related to an alcoholic formulation comprising a mixture of alkaloids, specifically piperine, piperettine, trichostaquine, piperoleins, other alkamides, and/or other alkaloids, as well as terpenoids, phenolics, prenol lipids, and flavonoids. The formulation is particularly rich in piperines, which are found in greater proportion compared to the other compounds. This unique composition exhibits anti-tumor activity in vitro, reducing tumors generated by orthotopic transplantation of tumors in animal models. Furthermore, it modulates the immune response by reducing the frequency of intratumoral immunosuppressive cells and increasing the frequency of cytotoxic T lymphocytes, thereby promoting tumor destruction and controlling metastasis.

### FIELD OF THE INVENTION

The present invention relates to a formulation comprising a mixture of alkaloids, specifically piperine, piperettine, trichostaquine, piperoleins; prenol lipids and flavonoids. More particularly, the present invention describes an alcoholic formulation in which piperines are found in a greater proportion than the other compounds present in the mixture.

Furthermore, the formulation of the present invention has cytotoxic activity and immunomodulatory action; additionally, it decreases glucose uptake when used in a cellular environment.

### BACKGROUND OF THE INVENTION

Alkaloids are a diverse group of naturally occurring organic compounds that are found in plants and have various biological effects. Piperine is an alkaloid found in black pepper and has been shown to have numerous beneficial properties, including anti-inflammatory and antioxidant effects. The present invention aims to provide a formulation that contains a mixture of alkaloids, specifically piperine, and other compounds that may have beneficial effects on health.

Prior art collectively demonstrates the existence of previous knowledge and innovation in the field of alcoholic compositions comprising a mixture of alkaloids. They highlight the potential benefits and need of formulation strategies associated with such compositions.

Particularly, the invention disclosed in patent document KR 20040003668 shows an alkaloid compound having excellent platelet aggregation inhibitory activity, and more particularly, to an alkaloid compound useful for the prevention and treatment of brain and cardiovascular diseases, showing the potential of such compositions in the medical field.

Document WO2009041787 also provides a pharmaceutical composition for the prevention and treatment of inflammatory or infectious diseases, comprising alkaloid compounds as active ingredients.

Document US20150190392 discloses a mixture of alkaloids for treating cancer and neurodegenerative conditions containing: isoquinoline, bisbenzylisoquinoline, biscoclaurine and bisisoquinoline.

U.S. Pat. No. 5,744,161 to provide a composition with a piperidine alkaloid, used to stimulate gastrointestinal absorption and systemic utilization of nutritional material.

Mitra et. al. and many other authors have described over the years, many therapeutic properties including anticancer potential of many alkaloids.

The present invention aims to provide a formulation that contains a mixture of alkaloids, specifically piperine, and other compounds that may have beneficial effects on health.

Particularly, the present invention has shown cytotoxic activity in cancer cell lines, exhibiting a promising antitumor effect due to the modulation of the intratumoral immune response.

### SUMMARY OF THE INVENTION

The present invention relates to a formulation comprising a mixture of alkaloids such as piperine, piperettine, trichostaquine, piperoleins, besides prenol lipids such as caryophyllene, and flavonoids such as vitexin-O-rhamnoside. More particularly, the present invention describes an alcoholic formulation in which alkaloids are found in a greater proportion than the other compounds present in the mixture.

Furthermore, the formulation of the present invention has cytotoxic activity and immunomodulatory action; additionally, it decreases glucose uptake when used in a cellular environment.

### BRIEF DESCRIPTION OF DRAWINGS

Some embodiments of the present invention are illustrated as an example and are not limited by the figures of the accompanying drawings.

FIG. 1. In vitro activity of the formulation of the invention. Cell count per cm² of cancer cell line type 1 after treatments.

FIG. 2. In vitro activity of the formulation of the invention in cancer cell line type 2 after treatments.

FIG. 3. In vitro activity of the formulation of the invention and cancer cell line type 3 after treatments.

FIG. 4. Population doubling times (PDT) are shown. Frequency of cancer cell line type 1 (D) cells in apoptosis (sum of early and late apoptosis) expressed as mean±SEM for three independent experiments.

FIG. 5. Population doubling times (PDT) are shown. Frequency of cancer cell line type 2 (E) cells in apoptosis (sum of early and late apoptosis) expressed as mean±SEM for three independent experiments.

FIG. 6. Frequency of cancer cell line type 1(F) cells with depolarized membrane evaluated by flow cytometry after treatments for 6 and 12 h.

FIG. 7. Frequency of cancer cell line type 2 (G) cells with depolarized membrane evaluated by flow cytometry after treatments for 6 and 12 h.

FIG. 8. Fold change of H2DCFDA MFI after the treatments with alcoholic formulation, P2Et extract (anti-oxidant control), or doxorubicin (pro-oxidant control) in all cell lines. Data of three independent experiments are shown. *p<0.05; **p<0.01; **p<0.001; ***p<0.0001.

FIG. 9. Fold change of 2-NBDG MFI after treatments in all cell lines. In all cases, fold change was determined using MFI of each treatment relative to vehicle (ethanol or DMSO). Data of three independent experiments are shown. *p<0.05; **p<0.01; **p<0.001; ***p<0.0001.

FIG. 10. In vivo treatment with the formulation of the invention delays tumor growth and metastasis. Tumor volume in cancer cell line type 1 tumor-bearing mice treated with each treatment.

FIG. 11. In vivo treatment with the formulation of the invention delays tumor growth and metastasis. Tumor volume in cancer cell line type 2 tumor-bearing mice treated with each treatment.

FIG. 12. Frequency circulating blasts in cancer cell line type 3 model.

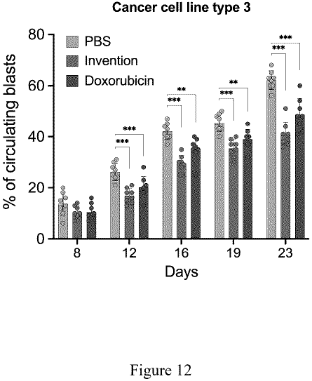

FIG. 13. Bars showing the percentage of mice that developed macrometastasis in murine model 1.

FIG. 14. Number of blast cells infiltrating organs. The p values were calculated using Kruskal-Wallis and Dunn's posttest for multiple comparisons. *p<0.05; **p<0.01; ****p<0.0001

FIG. 15. Results from experiments showing that the formulation of the invention modulates the tumor microenvironment reducing M-MDSC and PMN-MDSC. Frequency of cancer cell line type 1 intratumor CD45+ cells in mice treated with the alcoholic formulation, P2Et (positive control), or PBS (negative control).

FIG. 16. Overview of the immune cell composition in the model 1 TME shown in percentage of cells (out of CD45+ cells) on a per-mouse basis.

FIG. 17. Results from experiments showing that the formulation of the invention modulates the tumor microenvironment reducing M-MDSC and PMN-MDSC. opt-SNE visualization of clustering of some subpopulations from cell line type 1 tumor detected by flow cytometry.

FIG. 18. Results from experiments showing that the formulation of the invention modulates the tumor microenvironment reducing M-MDSC and PMN-MDSC. Frequency of activated CD8+ T cells (CD44+).

FIG. 19. Results from experiments showing that the formulation of the invention modulates the tumor microenvironment reducing M-MDSC and PMN-MDSC. Frequency of cancer cell line type 2 intratumor CD45+ cells in mice groups.

FIG. 20. Overview of the immune cell composition in the model 2 TME shown in percentage of cells (out of CD45+ cells).

FIG. 21. opt-SNE visualization of clustering of some immune subpopulations from cancer cell line type 2 tumor.

FIG. 22. Frequency of activated CD8+ T cells (CD44+). In all cases, data are represented as the mean±SEM. The p values were calculated using Mann-Whitney U test. *p<0.05; **p<0.01

FIG. 23. Results from experiments showing that the formulation of the invention modulates the immune response in lymph nodes. A. Overview of the immune cell composition in lymph nodes from cancer cell line type 1 tumor-bearing mice shown in percentage of cells on a permouse basis.

FIG. 24. Results from experiments showing that the formulation of the invention modulates the immune response in lymph nodes. Frequency of activated CD8+ T cells (CD44+). Data are represented as the mean±SEM. The p values were calculated using Mann-Whitney U test. *p<0.05; **p<0.01

FIG. 25. Results from experiments showing that the formulation of the invention modulates the immune response in lymph nodes. Overview of the immune cell composition in lymph nodes from cancer cell line type 2 tumor-bearing mice shown in percentage of cells on a per-mouse basis.

FIG. 26. Results from experiments showing that the formulation of the invention modulates the immune response in lymph nodes. Frequency of activated CD8+ T cells (CD44+). In all cases, data are represented as the mean±SEM. The p values were calculated using Mann-Whitney U test. *p<0.05; **p<0.01

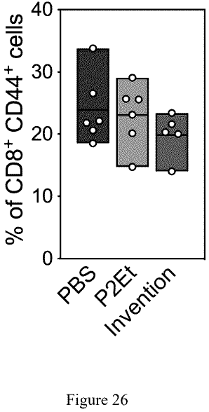

FIG. 27. Results showing that the formulation of the invention enhances the functional activity of the T cells in a cancer model. Functional activity of CD4+ in cell type 1. Each mice group determined using a five functions assay to measure simultaneous IFNγ, TNFα, IL-2, granzyme B, and perforin expression after stimulation with P/I. The functional profiles are grouped and color-coded according to the number of simultaneous T cell functions, as shown in the pie charts. Multifunctional analyzes were performed using a Boolean gating strategy with FlowJo v10.8.1 software and subsequently, data were analyzed and plotted with Pestle v2.0 and SPICE v6.1 software.

FIG. 28. Results showing that the formulation of the invention enhances the functional activity of the T cells in a cancer model type 1. Functional activity of CD8+ T-cells in murine model 1. Each mice group determined using a five functions assay to measure simultaneous IFNγ, TNFα, IL-2, granzyme B, and perforin expression after stimulation with P/I.

FIG. 29. Results showing that the formulation of the invention enhances the functional activity of the T cells in a cancer model. Functional activity of CD4+ in cell type 2 (C). Each mice group determined using a five functions assay to measure simultaneous IFNγ, TNFα, IL-2, granzyme B, and perforin expression after stimulation with P/I.

FIG. 30. Results showing that the formulation of the invention enhances the functional activity of the T cells in a cancer model. Functional activity of CD8+ T cells in murine model 2. Each mice group determined using a five functions assay to measure simultaneous IFNγ, TNFα, IL-2, granzyme B, and perforin expression after stimulation with P/I.

FIG. 31. Results showing that the formulation of the invention inhibits the PI3K/Akt/mTOR pathway. Fold change in frequency of cells presenting phosphorylated PI3K/AKT/mTOR proteins after treatments. Fold change was determined using the percentage of cells with phosphorylation of PI3K/AKT/mTOR proteins after each treatment relative to baseline (dashed line corresponds to normalization to experimental controls). P values were calculated using a nonparametric Mann-Whitney nonparametric test when comparing two groups. *p<0.05, **p<0.01, ***p<0.001, ****p<0.0001.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims.

The present invention provides a formulation comprising a mixture of metabolites such as alkaloids, prenol lipids and flavonoids. Still, it is not limited to, piperine, piperettine, trichostachine, piperoleins, other alkamides and/or another alkaloid, terpenoids and/or phenolics.

The alcoholic formulation has alkaloids 40-75%, prenol lipids 10-20%, and flavonoids 5-10%.

In a preferred embodiment the formulation is rich in alkaloids (40-75%) (Table 1), prenol lipids (10-20%), and flavonoids (5-10%) which are found in greater proportion than the other compounds in the mixture.

TABLE 1
Main alkaloids compounds in alcoholic formulation.

| | | | % (in relation to |
|---|---|---|---|
| | Molecular | Content (μg/mL of | total alkaloids |
| Compound | formula | formulation) | content) |
| Piperettine | C₁₉H₂₁NO₃ | <10.0 ± 0.06 | 3.2 |
| Trichostachine | C₁₆H₁₇NO₃ | <10.0 ± 0.27 | 2.2 |
| Piperine | C₁₇H₁₉NO₃ | 169.6 ± 0.94 | 54.6 |
| Not identified | C₁₉H₂₁NO₃ | 28.2 ± 0.80 | 8.7 |
| Not identified | C₁₇H₂₁NO₃ | 14.2 ± 0.36 | 4.1 |
| Piperolein A | C₁₉H₂₅NO₃ | 27.2 ± 0.79 | 8.3 |
| Piperolein B | C₂₁H₂₉NO₃ | 59.8 ± 0.69 | 18.9 |

The composition does not include artificial additives. The excipients would be included. The alcoholic formulation can be, in some embodiments, liquid and concentrated.

The formulation can be used in various applications, including as a drug, medical treatment, a dietary supplement or as an ingredient in food or beverage products. The formulation may provide various health benefits, including anti-inflammatory and antioxidant effects, and may also improve the taste and aroma of food and beverages.

The formulation of the present invention can be formulated orally, topically, or intravenously. For oral administration, the formulation can be mixed with a suitable excipient or carrier to form a tablet, capsule, or liquid suspension. For topical administration, the formulation can be applied directly to the affected area. For intravenous administration, the formulation can be injected directly into the bloodstream.

The present invention provides a unique and innovative formulation that contains a mixture of alkaloids, specifically piperine, and other compounds that may have beneficial effects on health. The formulation may have applications in various industries, including the medical, food and beverage industry, and may provide a safer and more effective alternative to traditional chemotherapy and to dietary supplements.

The present invention relates to an alcoholic formulation comprising a mixture of alkaloids with antitumoral activity.

The alkaloid formulation has cytotoxic activity and, additionally, decreases glucose uptake.

The combined alkaloid formulation is more cytotoxic than the main alkaloids, piperine, and has better specificity for tumor cells than the isolated compounds.

The formulation has an antitumor effect that decrease the frequency of blasts, a longer survival compared to a control group and a lower infiltration of tumor cells in the bone marrow, spleen and lung compared to a control group.

Moreover, the formulation of the invention exerts immunomodulatory functions reducing the immunosuppressive microenvironment, slightly related to the modulation of cellular energy metabolism.

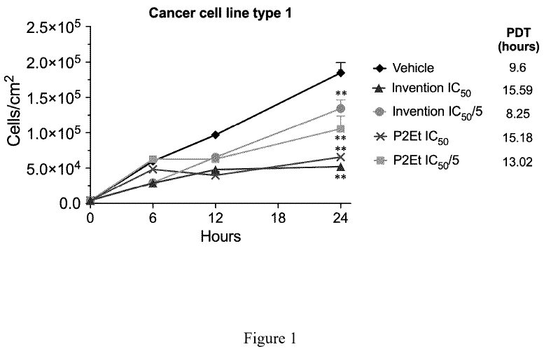

The formulation of the present invention, when used in cancer cell lines presents cytotoxic activity against different cell lines at low concentrations (FIG. 1).

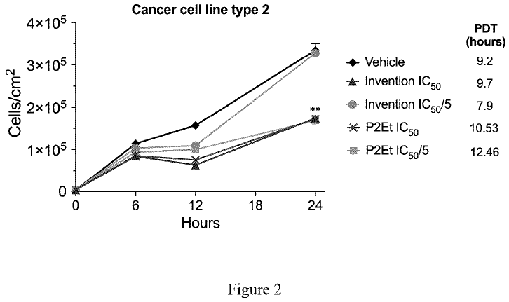

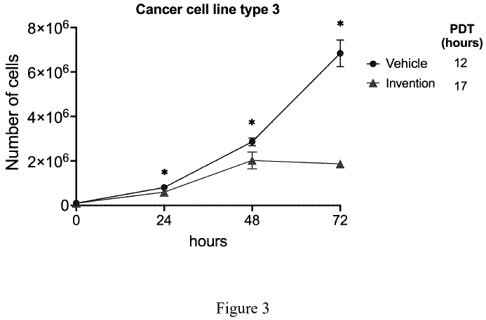

In addition, the formulation of the invention significantly reduced the proliferation of tumor cell line type 1 (FIG. 1), cancer cell line type 2 (FIG. 2), and cancer cell line type 3 (FIG. 3) at both IC₅₀ and ⅕ of the IC₅₀ in the first 24 hours.

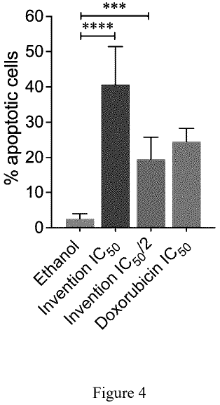

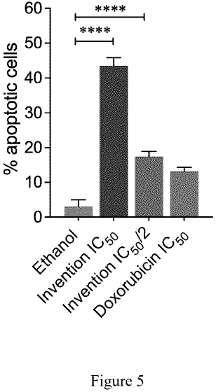

Then, when evaluating the type of tumor cell death induced by the formulation of the invention it was found that the treatment for 24 h induces apoptosis in both tumor cell line type 1 (FIG. 4) and cancer cell line type 2 (FIG. 5) cells.

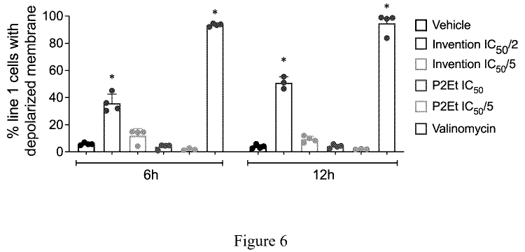

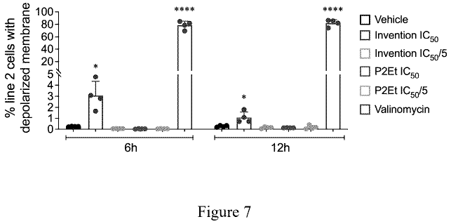

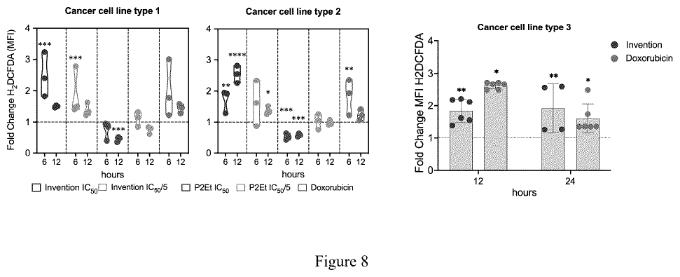

In addition, tumor cell line type 1 (FIG. 6) and cancer cell line type 2 (FIG. 7) treated with the formulation of the invention at different concentrations for 6 and 12 h showed mitochondrial membrane depolarization with the highest dose at both treatment time. According to this result, the formulation of the invention also presented pro-oxidant activity in the cell lines. However, while in tumor cell line type 1 it decreases over time, in cancer cell line type 2 it increases at 12 hours with the IC₅₀, suggesting a difference of the mechanisms activated by the formulation of the invention and involved in ROS induction (FIG. 8).

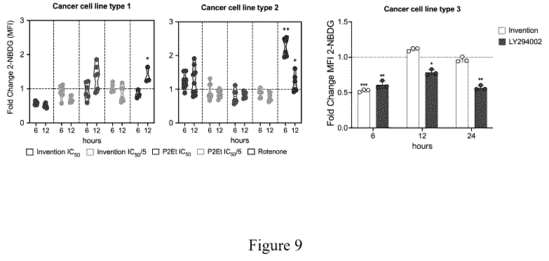

Moreover, regarding the effect of the formulation on glucose uptake in the cancer cell line type 2, it induces a dose-dependent decrease in intracellular glucose uptake, in contrast to a small increase in intracellular glucose uptake observed in cancer cell line type 2 (FIG. 9).

In this embodiment, the formation of the invention has tumor specificity and has shown differential effect in various cancer lines and models.

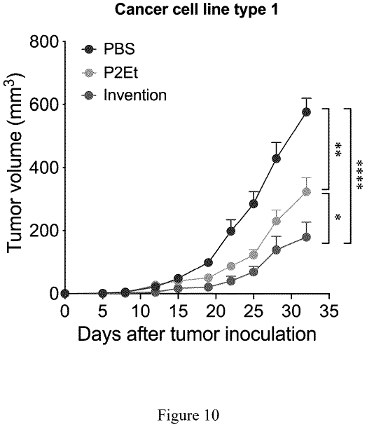

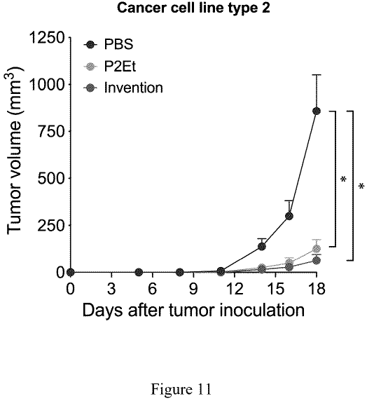

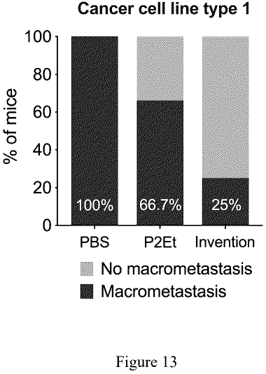

Other in vivo experiments, show that the formulation of the invention significantly decreased tumor size in a tumor cell line type 1 model (FIG. 10), as well as in cancer cell line type 2 model (FIG. 11), in the same way as a positive control P2Et. Likewise, with the type 3 cancer cell line, a decrease in tumor development like that observed with the positive control doxorubicin was found (FIG. 13).

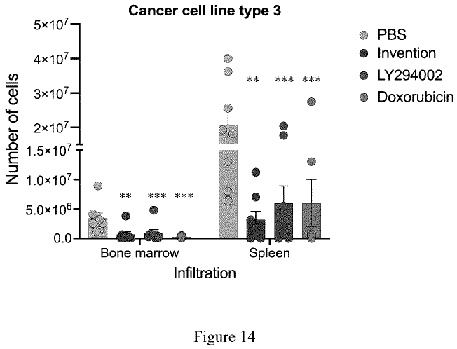

Interestingly, it was also observed that metastasis of tumor cell line type 1 model (FIG. 2D) and tumor cell line type 3 model (FIG. 14) were controlled to a greater extent by the formulation of the invention than even by the positive control.

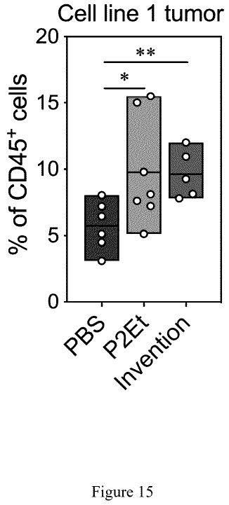

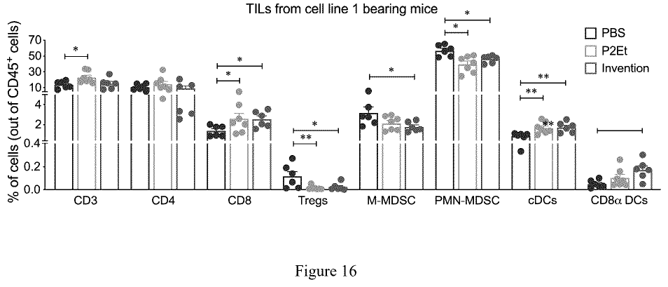

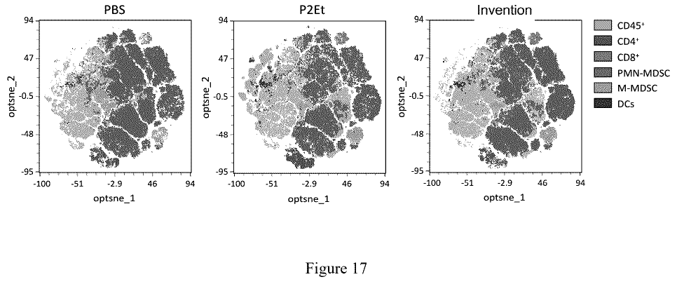

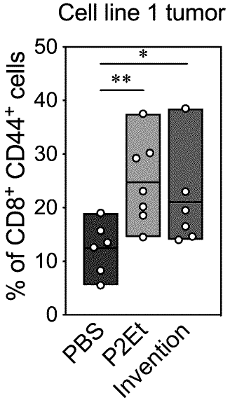

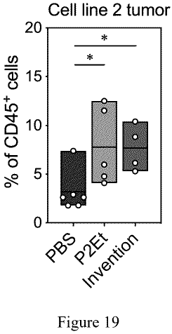

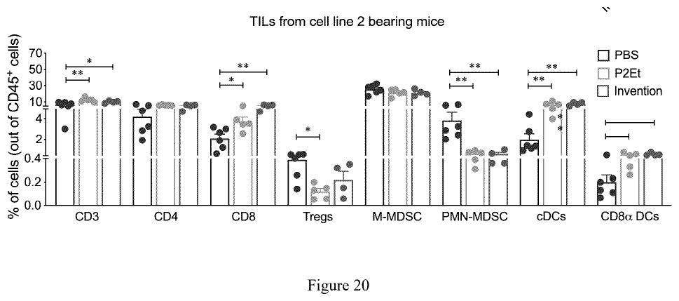

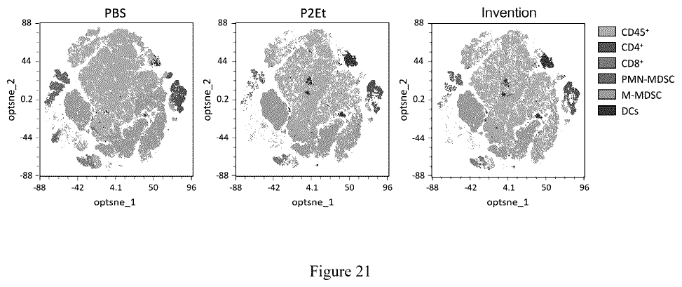

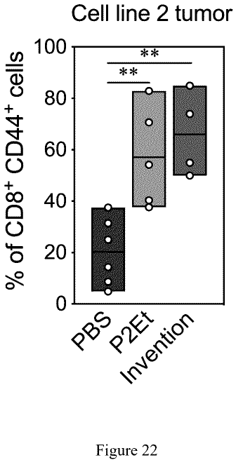

Moreover, the antitumor immune response induced by the formulation of the invention reveals an increase in the infiltrate of CD45+ hematopoietic cells, just as P2Et extract does in both cell lines (FIGS. 15 and 17). In the case of tumor cell line type 1 model, when treated with formulation of the invention presented an infiltrate composed mainly of activated CD8+ T cells (CD44+), conventional dendritic cells (cDC) and CD8+DC, with a significant decrease in Treg, M-MDSC-LC and PMN-MDSC-LC (FIGS. 16 and 18). In cancer cell line type 2 tumors, an increase in activated CD8+ T cells (CD44+) (FIG. 19), as well as cDC and CD8+DC, and a decrease in PMN-MDSC-LC were also observed (FIG. 20), but unlike what was observed in the tumor cell line type 1, the decrease in regulatory T cells (Tregs) was not significant, and a decrease in M-MDSC-LC was not observed (FIGS. 21 and 22).

These results suggest that in both cases, there is an activation of the adaptive immune response evidenced by the activation of CD8+ T cells, and the migration of CD8+DC to the tumor lumen, which are mainly involved in intratumoral cross priming.

However, a differential regulation of the tumor microenvironment in different cell lines was also evident (FIGS. 17 and 21).

Moreover, this differential effect can be useful in different cancer types with slightly different functional mechanisms induced by the formulation of the invention. In this same embodiment, at a molecular level it could be possible to envision these results to other cancer models with similar molecular behaviors.

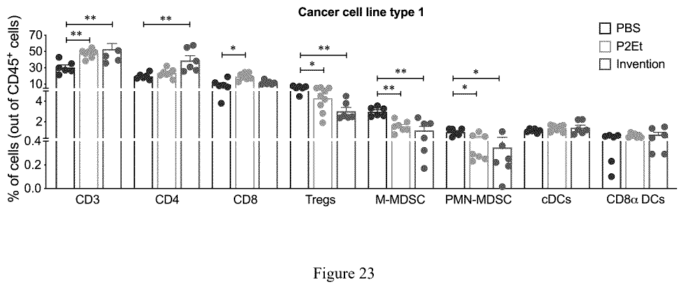

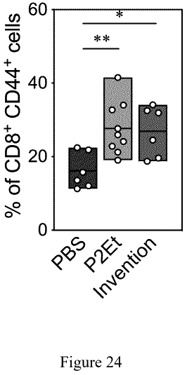

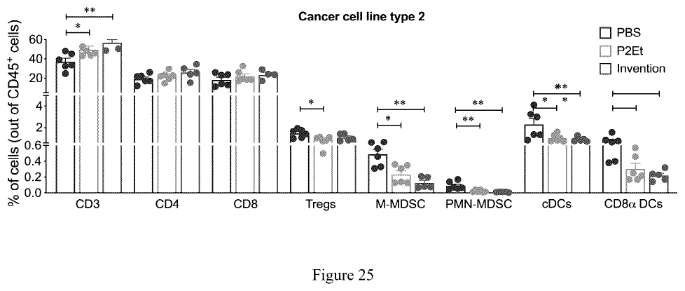

Further analysis in vivo models also showed that the formulation of the invention induces a systemic immune response, showing a higher frequency of CD4+ T cells and a lower frequency of Treg, M-MDSC-LC and PMN-MDSC-LC in infiltrates (FIGS. 23 and 25). In addition, an increase in the frequency of activated CD8+ T cells was found (FIG. 24).

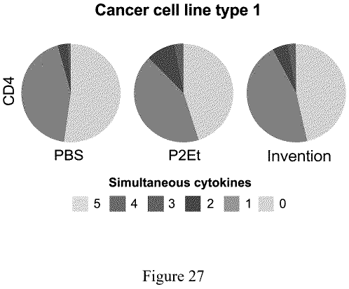

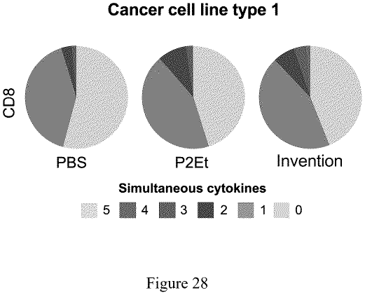

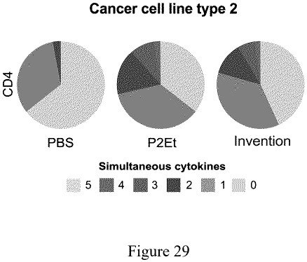

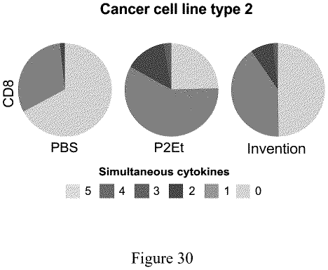

The formulation of the invention induces a higher frequency of multifunctional CD4+(FIGS. 27 and 29) and CD8+ T cells (FIGS. 28 and 30), compared to the control group, like that observed with group treated with P2Et.

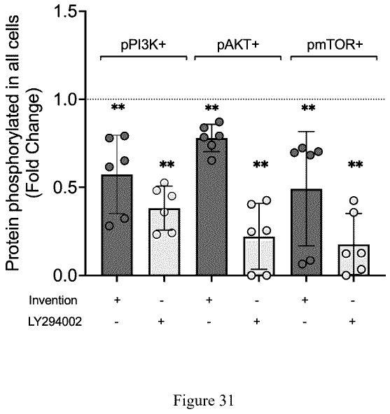

The formulation of the invention inhibits the PI3K/AKT/mTOR pathway, which plays a crucial role in cancer development and progression. This pathway is often hyperactivated in many types of cancer, promoting cell growth, proliferation, and survival. By inhibiting this pathway, the invention has the potential to suppress tumor growth and metastases development (FIG. 31).

In this document the P2Et compound used in experiments corresponds to formulations in US20230190848.

1. Alcoholic formulation with a mixture of alkaloids, prenol lipids and flavonoids, wherein:
a) the mixture of alkaloids comprises piperine, piperettine, trichostaquine, and piperoleins A and B;
b) the mixture of prenol lipids comprises caryophyllene, humulene, bisabolene;
c) the mixture of flavonoids comprises vitexin-O-rhamnoside, vicenin, kaempferol-rhamnoside-glucoside.

2. The alcoholic formulation of claim 1, wherein alkaloids are between 40-75%, prenol lipids 10-20%, and flavonoids 5-10%.

3. The alcoholic formulation of claim 1, wherein the piperine content is at least 50% of the total alkaloid content in the formulation.

4. The alcoholic formulation of claim 1, wherein the mixture of prenol lipids also comprises any of: linalool, terpineol, elemene, famesene, selinene, farnesol, alphatocopherol, abscisic acid or mixtures of these.

5. The alcoholic formulation of claim 1, wherein the mixture of flavonoids also comprises any of: rhoifolin, orientin, baicalein, or mixtures of these.

6. The alcoholic formulation of claim 1, wherein the formulation does not include artificial additives.

7. The alcoholic formulation of claim 1, wherein the formulation exhibits cytotoxic activity and decreases glucose uptake when tumor cells are treated.

8. The alcoholic formulation of claim 1, wherein the formulation exhibits antitumoral activity.

9. The alcoholic formulation of claim 1, wherein the formulation exerts immunomodulatory functions like diminution of immunosuppressive microenvironment.

10. The alcoholic formulation of claim 1, wherein the formulation decreases proliferation of tumor cell lines in a dose-dependent manner.

11. The alcoholic formulation of claim 1, wherein the formulation induces apoptotic cell death and has a prooxidant effect on tumor cell lines.

12. The alcoholic formulation of claim 1, wherein the formulation significantly reduces tumor size in tumor models.

13. The alcoholic formulation of claim 1, wherein the formulation significantly increases the frequency of dendritic cells and activated CD8+ T cells and decreases the frequency of MDSCs and Tregs in the tumor microenvironment of tumor models.

14. The alcoholic formulation of claim 1, wherein the formulation increases the frequency of T cells producing IFNg, TNFa, and IL-2, and increases the frequency of multifunctional T cells in tumor models.

15. Use of alcoholic composition of claim 1 for inhibits the activation of the PI3K/Akt/mTOR signaling pathway, thereby reducing downstream cellular processes associated with proliferation, survival, growth, cellular migration and cancer metastasis.
